(19) United States
(12) Patent Application Publication
(10) Pub. No.: US 2007/0179481 A1
(43) Pub. Date: Aug. 2, 2007

(54) Laser System for Treatment of Skin Laxity

(75) Inventors: George Frangineas, Fremont, CA (US); Leonard C. DeBenedictis, Palo Alto, CA (US); Thomas R. Myers, Palo Alto, CA (US); Basil M. Hantash, East Palo Alto, CA (US)

Correspondence Address: RELIANT / FENWICK;c/o FENWICK & WEST, LLP, 801 CALIFORNIA STREET, MOUNTAIN VIEW, CA, 94041, US

(73) Assignee: Reliant Technologies, Inc., Mountain View, CA (US)

(21) Appl. No.: 11/674,654

(22) Filed: Feb. 13, 2007

Related U.S. Application Data
(63) Continuation-in-part of application No. 10/367,582, filed on Feb. 14, 2003.
(63) Continuation-in-part of application No. 10/888,356, filed on Jul. 9, 2004.
(60) Provisional application No. 60/773,192, filed on Feb. 13, 2006.

(51) Int. Cl. A61B 18/18

(52) U.S. Cl. 606/9

(57) ABSTRACT

A method of reducing wrinkles in skin includes irradiating the skin with laser pulses to ablate an array of spaced apart voids in the skin. A region of coagulated tissue surrounds each of the voids and there viable tissue between the coagulated regions. Tissue in the coagulated regions is in tension due to shrinkage of collagen by the heat generated during the ablation. This tension rapidly closes the voids, tightening the skin and reducing the wrinkles. A healing process replaces the coagulated tissue with new tissue after a period of about one-month. The method is also applicable to lightening abnormally pigmented skin, as the new tissue replacing the coagulated tissue is not abnromally pigmented.

### CROSS-REFERENCE TO RELATED APPLICATION(S)

This application is (a) a continuation-in-part of U.S. patent application Ser. No. 10/367,582, “Method and Apparatus for Treating Skin Using Patterns of Optical Energy,” filed Feb. 14, 2003, (b) a continuation-in-part of U.S. patent application Ser. No. 10/888,356, “Method and Apparatus for Fractional Photo Therapy of Skin,” filed Jul. 9, 2004, and (c) claims priority under 35 U.S.C. 119(e) to U.S. Provisional Patent Application Ser. No. 60/773,192, “Laser System for Treatment of Skin Laxity,” filed Feb. 13, 2006. The subject matter of all of the foregoing is incorporated herein by reference in its entirety.

### TECHNICAL FIELD OF THE INVENTION

The present invention relates in general to laser dermal treatment, including for example methods of cosmetic treatment for skin tightening and wrinkle reduction by laser irradiation.

### DISCUSSION OF BACKGROUND ART

The aesthetic treatment of skin for rejuvenation purposes including skin tightening for wrinkle reduction and the like has hitherto involved primarily the removal of tissue and subsequent wound healing to effect the treatment. Chemical peels, dermabrasion, and ablative laser skin resurfacing are used routinely for this purpose. Such treatments usually involve some degree of discomfort, and with more aggressive treatments there can be a risk of injury. Further, these treatments typically leave large open wounds which must subsequently heal. Accordingly there can be a “down time” period as long as several weeks, during which treated skin may have a worse appearance than before the treatment, before positive results of the treatment appear.

Generally the effectiveness of ablative laser treatments for wrinkle reduction is proportional to the down time, discomfort and risk induced by the treatment. There is need for a wrinkle reduction treatment that results in deep remodeling of the skin to provide long term wrinkle reduction by skin tightening but does not have the down time associated with prior art ablative laser treatments.

### SUMMARY OF THE INVENTION

The present invention is directed to a method of tightening human skin characterized as having a dermal layer (dermis) surmounted by an epidermal layer (epidermis) surmounted in turn by an outer, stratum corneum layer. The method, which may be used for cosmetic or non-cosmetic purposes, comprises irradiating the skin with laser radiation in a manner such that a plurality of elongated voids of particular spatial frequency is formed in the skin. The voids extend through the stratum corneum, through the epidermis, and into the dermis, with walls of the voids being cauterized by the laser radiation, with a volume of coagulated dermal tissue surrounding the voids, and with viable epidermal and dermal tissue remaining between the coagulated tissue surrounding the voids. Tension in the coagulated tissue shrinks the voids, thereby tightening the skin. A wound-healing response that is enhanced by adjacent viable tissue causes replacement of the coagulated tissue with new viable tissue, thereby further tightening the tissue and enhancing the tissue elasticity.

The method of the present invention may be described as a fractional ablative treatment. This fractional ablative treatment allows for volume removal of tissue with fewer side effects than would be possible with broad-area, i.e., non-fractional treatment. The viable tissue between the regions of coagulated tissue surrounding the voids allows the wound healing process to respond efficiently to the laser treatment, due to the presence of viable tissue to orchestrate this response. For effective treatment, this-sparing of normal viable tissue between ablated voids must take place. This, together with sharp temperature-profile gradients characteristic of the inventive fractional ablation, spares proteins and pathways in a significant fraction of the wound. The sparing of proteins and pathways enables protein activity that is important to the wound-healing response.

### BRIEF DESCRIPTION OF THE DRAWINGS

The accompanying drawings, which are incorporated in and constitute a part of the specification, schematically illustrate a preferred embodiment of the present invention, and together with the general description given above and the detailed description of the preferred embodiment given below, serve to explain principles of the present invention.

FIG. 1 is a micrograph of a section of human skin immediately after irradiation with laser radiation having parameters in accordance with the method of the present invention, the irradiated skin including a plurality of voids extending through the stratum corneum and the epidermis into the dermis, the voids being surrounded by regions of coagulated dermal tissue with viable tissue between the regions of coagulated tissue surrounding the voids.

FIG. 2 is a micrograph similar to the micrograph of FIG. 1 but having a lower magnification and depicting detail of the voids extending through the stratum corneum.

FIG. 3 is a micrograph of a section of human skin 48 hours after irradiation with laser radiation having the parameters in accordance with the method of FIG. 1.

FIG. 4 is a micrograph of a section of human skin one week after irradiation with laser radiation having the parameters in accordance with the method of FIG. 1.

FIG. 5 is a micrograph of a section of human skin one-month after irradiation with laser radiation having the parameters in accordance with the method of FIG. 1.

FIG. 6 is a graph schematically illustrating trend curves for maximum lesion or treatment zone width (void width plus coagulated tissue width) as a function of lesion or zone depth in the method of the present invention, for 5 mJ, 10 mJ, and 20 mJ pulses.

FIG. 7 is a graph schematically illustrating trend curves for maximum void width as a function of lesion or zone depth in the method of the present invention, for 5 mJ, 10 mJ, and 20 mJ pulses.

FIGS. 8A, 8B, and 8C are graphs schematically illustrating estimated width as a function of lesion or zone depth for lesions and voids with dimensions derived from micrographs of treatment sites in accordance with the present invention, for respectively 5 mJ, 10 mJ, and 20 mJ pulses.

FIG. 9A is a front elevation view schematically illustrating one example of apparatus suitable for irradiating skin according to the method of the present invention, the apparatus including a multi-faceted scanning wheel for scanning a pulsed, collimated laser beam and a wide field lens for focusing the scanned laser beam onto skin to sequentially ablate tissue and create the cauterized voids of the inventive method.

FIG. 9B is a front elevation view schematically illustrating further detail of beam focusing in the apparatus of FIG. 9A.

FIG. 9C is a side elevation view schematically illustrating still further detail of beam focusing in the apparatus of 9A.

FIG. 10 schematically illustrates detail of the scanning wheel of FIGS. 9A-C.

FIG. 11 schematically illustrates one example of a handpiece including the apparatus of FIGS. 9A-C, the handpiece including a removable tip connectable to a vacuum pump for exhausting smoke and ablation debris from the path of the laser beam.

FIGS. 12A, 12B, 12C, and 12D are micrographs of sections of human skin excised from the forearms of human subjects after irradiation with laser radiation having parameters in accordance with the method of the present invention, the irradiated skin including a plurality of voids extending through the stratum corneum and the epidermis into the dermis, the voids being surrounded by regions of coagulated dermal tissue with viable tissue between the regions of coagulated tissue surrounding the voids. The lesions were produced in vivo and biopsied within 1 hour following irradiation. Treatment energies used were (A) 5 mJ, (B) 10 mJ, (C) 20 mJ, and (D) 30 mJ.

FIGS. 13A, 13B, 13C, and 13D are micrographs similar to the micrograph of FIG. 12A, but each created with a treatment energy of 20 mJ and excised at 2 days, 7 days, 1 month, and 3 months, respectively, following laser irradiation.

FIGS. 14A and 14B are images of a single micrograph of human skin excised from the forearm of a human subject after irradiation with laser irradiation having parameters in accordance with the method of the present invention, the irradiated skin including a plurality of voids extending through the stratum corneum and the epidermis into the dermis, the voids being surrounded by regions of coagulated dermal tissue with viable tissue between the regions of coagulated tissue surrounding the voids. The lesion was produced in vivo and biopsied following laser irradiation. The micrograph was taken from the papillary dermis and photographed using unpolarized (FIG. 14A) and cross-polarized (FIG. 14B) microscopy. FIGS. 14A and 14B show an ablated zone surrounded by an annular coagulation zone. The cross-polarized image indicates the loss of birefringence, confirming the denaturation of the collagen matrix within the coagulation zone.

### DETAILED DESCRIPTION OF THE PREFERRED EMBODIMENTS

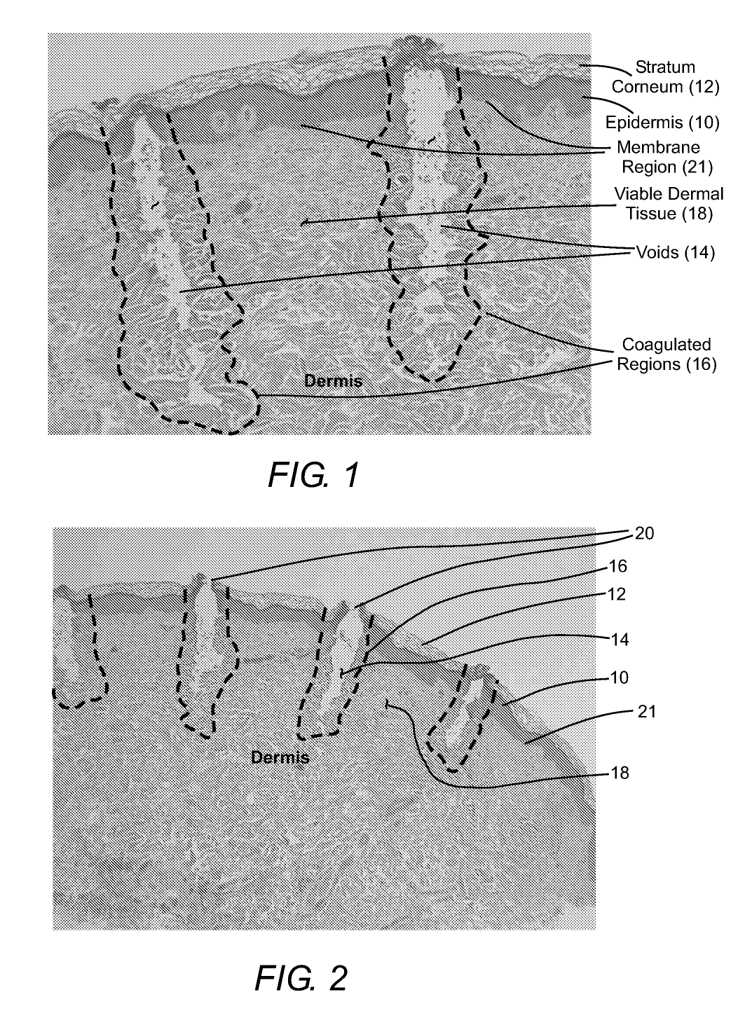

Referring now to the drawings, wherein like features are designated by like reference numerals, FIG. 1 and FIG. 2 are micrographs schematically illustrating a section of human skin immediately after irradiation with laser radiation having parameters in accordance with the method of the present invention. FIG. 2 is at twice the magnification of FIG. 1. The skin was irradiated at spaced-apart locations with pulses of radiation having a wavelength of 10.6 micrometers (μm) from a CO₂ laser delivering a substantially TEM₀₀-quality beam. Each location was irradiated by one pulse, although multiple pulses could be used in alternate embodiments. The radiation at the locations was focused to a spot having a diameter of about 120 μm at the surface of the skin, expanding slightly to between about 150 μm and 170 μm at a depth of about 1 mm in the skin. The laser output was repetitively pulsed at a pulse repetition frequency (PRF) of about 60-100 Hz. The pulses were nominally “square” laser pulses having a peak power of about 40 Watts (W) and a pulse duration of about 0.5 milliseconds (ms) to produce a pulse energy of 20 milljoules (mJ). The pulse duration could be varied to create different pulse energies for other experimental treatments. Experimental evaluations were performed with pulse energies in a range between about 5 mJ and 40 mJ. Laser pulses were scanned over the surface using a scanner wheel device to provide the spaced apart voids. The PRF of the laser was synchronized with the rotation of the scanner wheel. A detailed description of a preferred example of such a scanner wheel is presented further hereinbelow.

The skin tissue includes a bulk dermal portion or dermis covered by an epidermal layer (epidermis) 10 typically having a thickness between about 30 μm and 150 μm. The top layer of the epidermis is a stratum corneum layer 12 typically having a thickness between about 5 μm and 15 μm. Tissue was ablated at each pulse location, producing a plurality of spaced-apart voids 14, elongated in the direction of incident radiation, and extending through the stratum corneum and the epidermis into the dermis.

In the example of FIGS. 1 and 2, the voids with the parameters mentioned above have an average diameter (width) of between about 180 μm and 240 μm. These dimensions are provided merely for guidance, as it will be evident from the micrographs that the diameter of any one void varies as the result of several factors including, for example, the inhomogeneous structure and absorption properties of the tissue. The voids have an average depth of between about 800 μm and 1000 μm, and are distributed with a density of approximately 400 voids per square centimeter (cm²). Walls of the voids are substantially cauterized by heat generated due to the ablation, thereby mimimizing bleeding into and from the voids. This heat also produces a region 16 of coagulated tissue (coagulum) surrounding each void. The void is the region that is ablated. Immediately following ablation the voids typically are open at the surface. The appearance of closure of some voids in FIG. 2 is believed to be an artifact of the preparation of tissue samples for microscopic evaluation or an artifact of the angle of slicing through the tissue.

The coagulated regions have a thickness between about 20 μm and 80 μm immediately after ablation of the voids. Here again, however, thickness varies randomly with depth of the void because of above-mentioned factors affecting the diameter of the void. Between each void 14 and the surrounding coagulum 16 is a region 18 of viable tissue that includes a viable region of the epidermis and the dermis. Preferably the region of viable tissue has a width, at a narrowest point thereof, at least about equal to the maximum thickness of the coagulated regions 16 to allow sufficient space for the passage of nutrients to cause rapid healing and to preserve an adequate supply of transit amplifying cells to perform the reepithelialization of the wounded area. More preferably, the viable tissue separating the coagulated tissue around the voids has a width, at a narrowest point thereof, between about 50 μm and 500 μm. A preferred density of treatment zones is between about 200 and 5000 treatment zones per cm² and more preferably between about 1000 and 3000 treatment zones per cm². This treatment-zone density can be achieved in a single pass or multiple passes of a treatment device or applicator, for example two to ten passes, in order to minimize gaps and patterning that may be present if treatment zones are created in a single pass of the applicator.

Heat from the ablation process that causes the coagulation in regions 16 effectively raises the temperature of the collagen in those coagulated regions sufficiently to create dramatic shrinkage or shortening of collagen in the coagulated tissue. This provides a hoop of contractile tissue around the void at each level of depth of the void. Upon collagen shrinkage, the dermal tissue is pulled inward, effectively tightening the dermal tissue. This tightening pulls taut any overlying laxity through a stretching of the epidermis and stratum corneum. This latter response is primarily due to the connection of a basement membrane region 21 of the epidermis to the collagen and elastin extra-cellular matrix. This connection provides a link between the epidermis and dermis. The contractile tissue very quickly shrinks the void, and creates an increase in skin tension resulting in a prompt significant reduction in overall skin laxity and the appearance of wrinkles. This shrinkage mechanism is supplemented by a wound-healing process described below.

Closure of the void occurs within a period of about 48 hours or less through a combination of the above-described prompt collagen shrinkage and the subsequent wound healing response. The wound healing process begins with re-epithelialization of the perimeter of the void, which typically takes less than 24 hours, formation of a fluid filled vacuole, followed by infiltration by macrophages and subsequent dermal remodeling by the collagen and elastin forming fibroblasts. The column of coagulated tissue has excellent mechanical integrity that supports a progressive remodeling process without significant loss of the original shrinkage. In addition, the coagulated tissue acts as a tightened tissue scaffold with increased resistance to stretching. This further facilitates wound healing and skin tightening. The tightened scaffold serves as the structure upon which new collagen is deposited during wound healing and helps to create a significantly tighter and longer lasting result than would be created without the removal of tissue and the shrinkage due to collagen coagulation.

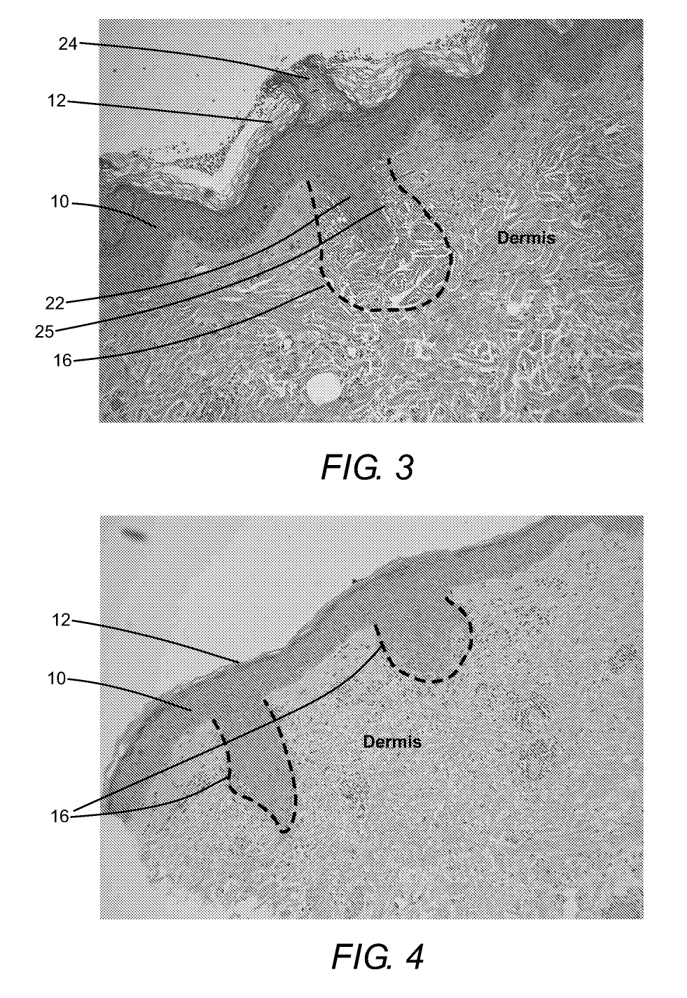

Progress of the healing after a period of about 48 hours from the irradiation conditions of FIG. 1 is illustrated by the micrograph of FIG. 3, which has the same magnification. Here, the coagulated region 16 is reduced both in diameter and depth compared with a comparable region of FIG. 1. In the micrograph of FIG. 3 epidermal stem cells have migrated into the void and facilitated healing of the void area. The healing response includes the release of heat shock proteins. This leads to the initiation of a regenerative cascade that includes integration of heat shock signals and subsequent release of growth factors such as FGF, angiogenesis factors such as VEGF, chemotactic factors such as IL-8, and contractile factors such as TGF-beta. Epidermal stem cells proliferate and differentiate into epidermal keratinocytes filling the void in a centripetal fashion. As epidermal cells proliferate and fill the void, the coagulated material is pushed up the epidermis toward the stratum corneum. The voids contain microscopic-epidermal necrotic debris (MEND). The pushing of the coagulated material forces a plug 24 of the MEND to seal the stratum corneum during the healing response, thus limiting access of the outside environment to the inside of the skin.

At this time, the basement membrane is ill-defined and has yet to be completely repaired and restored. This is clearly depicted by the vacuolar space 25 separating the healed void and the dermis. In FIGS. 1 and 2, there is sparse cellularity evident in the dermis. However, in the micrograph of FIG. 3, the wound healing response at 48 hours has led to increased release of signaling molecules, such as chemokines, from the area of spared tissue, leading to recruitment of inflammatory cells promoting the healing response.

Progress of the healing after a period of about one week from the irradiation conditions of FIG. 1 is illustrated by the micrograph of FIG. 4. Here, the MEND has been exfoliated. The void has been replaced by epidermal cells which gradually remodel the epidermis to create a normal rete ridge pattern, reducing the depth of invagination. The healing process has triggered some of the deeper epidermal cells to go through apoptosis, thereby disappearing from the replaced void tissue. The basement membrane of the epidermis has been almost fully restored as evidenced by the lack of vacuolization between the epidermis and dermis. During the wound healing response, cytokines such as TGF beta, amongst others, are released and stimulate fibroblast secretion of collagen, elastin, and extracellular matrix. This secreted matrix replaces the dermal component of the void. Inflammatory cells also help remove non-viable debris in the dermis, allowing the replacement of coagulated tissue with fresh viable tissue as outlined above.

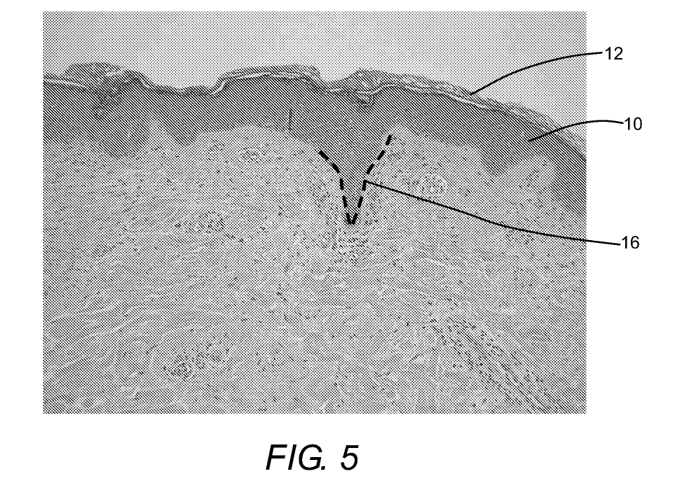

FIG. 5 depicts progress of healing one-month after initial treatment. Here remodeling of the void has continued by apoptosis of the deeper epidermal cells, leading to a more natural rete ridge like structure. The MEND is absent, and the basement membrane of the epidermis is completely healed. Inflammatory cells are still present in the dermis, and fibroblasts continue to lay down new matrix in the dermis. This provides that over the ensuing two to six months, new collagen synthesis continues to replace previously coagulated dermal tissue, providing for increased tensile strength in the dermis.

The complete replacement of the coagulated tissue providing the initial skin tightening with new collagen and elastin deposition as described above provides for a long lasting improvement in the appearance of wrinkles in temporally or photo aged skin. As the inventive method results in a completely healthy treated area once the healing process is complete, an area of skin treated once can be treated again, for example, after a period of about one week to two months to provide further improvement. Clearly, however, the progress of skin aging and loosening cannot be arrested permanently, and the length of time that any improved appearance will be evident will depend on the age of the person receiving the treatment and the environment to which treated skin is exposed, among other factors.

In the example described above, skin irradiation for void formation is performed with laser radiation having a wavelength (10.6 μm) that is strongly absorbed by water. Preferably the radiation is delivered as a beam having TEM₀₀ quality, or near TEM₀₀ quality. The CO₂ laser used in the example of the present invention discussed above is a relatively simple and relatively inexpensive laser for providing such a beam. The 10.6 μm radiation of a CO₂ laser has an absorption coefficient in water of approximately 850 inverse centimeters (cm⁻¹). To efficiently ablate tissue based on absorption in water, a high absorption coefficient in the water of the skin tissue is desired. However, in order to form a coagulation region surrounding the voids, to cause tissue shrinkage and to reduce bleeding at the treatment sites, the absorption coefficient should not be too high. If void creation is based on absorption in water, laser radiation used in the inventive method should have an absorption coefficient in water in the range between about 100 cm⁻¹ and 12,300 cm⁻¹. More preferably, the absorption coefficient should be between about 100 cm⁻¹ and 1000 cm⁻¹ and more preferably in the range between about 500 cm⁻¹ and 1000 cm⁻¹. In each of these absorption levels, laser pulses for forming the voids preferably have a duration between about 100 microseconds (μs) and 5 ms. The actual treatment parameters can be chosen based on commercial tradeoffs of available laser powers and desired treatment-zone sizes. Lasers providing radiation having a wavelength that has an absorption coefficient in water in the preferred ranges include CO₂, CO, and free-electron lasers (absorption coefficients in water of 500-1000 cm⁻¹), thulium-doped fiber lasers, Raman-shifted erbium-doped fiber lasers, and free-electron lasers (100-1000 cm⁻¹), Er:YAG lasers, and free-electron lasers (between about 100 cm⁻¹ and 12,300 cm⁻¹). Other light sources, such as optical parametric oscillators (OPOs) and laser pumped optical parametric amplifiers (OPAs) can also be used.

Voids 14 preferably have a diameter between about 100 μm and 500 μm, and are preferably spaced apart with a center to center distance of between about 200 μm and 1500 μm depending on the size of the voids 14 and the coagulated regions 16. The center to center distance can be chosen based on the level of desired treatment. A coverage area for the coagulated regions and voids immediately following treatment is preferably between about 5% and 50% of the treated area. A higher level of coverage will be more likely to have a higher level of side effects for a similar treatment energy per treatment site. A preferred depth of the voids is between about 200 μm and 4.0 millimeters (mm). The voids are preferably randomly distributed over an area of skin being treated.

In relative and practical terms, the voids are preferably placed such that coagulated zones 16 surrounding the voids are separated by at least the average thickness of the coagulated zones. This can be determined by making micrographs of test irradiations, similar to the above-discussed micrographs of FIGS. 1 and 2. If voids are too closely spaced, the healing process may be protracted or incomplete. If voids are spaced too far apart, more than one treatment may be necessary to achieve an acceptable improvement. Regarding depth of the voids, the voids and surrounding coagulated zones must extend into the dermis in order to provide significant skin tightening.

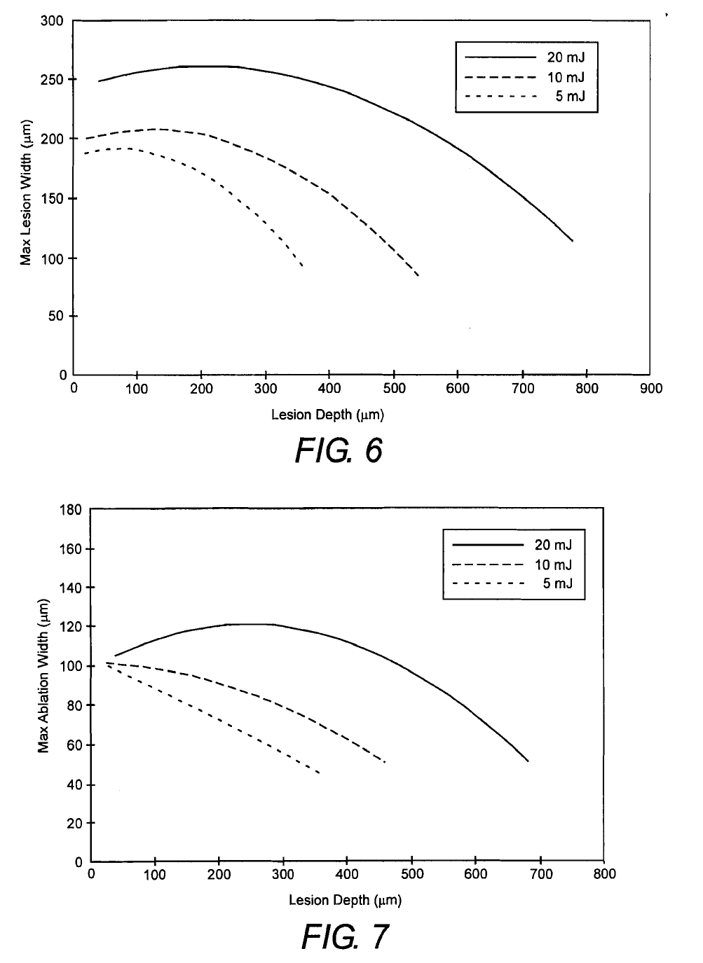

FIG. 6 and FIG. 7 are graphs schematically illustrating respectively trends for maximum width of the treatment zone (lesion), i.e., maximum total width of a void 14 plus surrounding coagulated region 16, and maximum width of the void (ablated region), as a function of lesion depth, i. e., the depth to the base of the coagulated region. The trends in each graph are shown for pulse energies of 5 mJ, 10 mJ, and 20 mJ. It should be noted here that these trends were fitted through a number of experimental measurements with relatively wide error bars, particularly at shallow lesion depth. Accordingly, it is recommended that these graphs be treated as guidelines only.

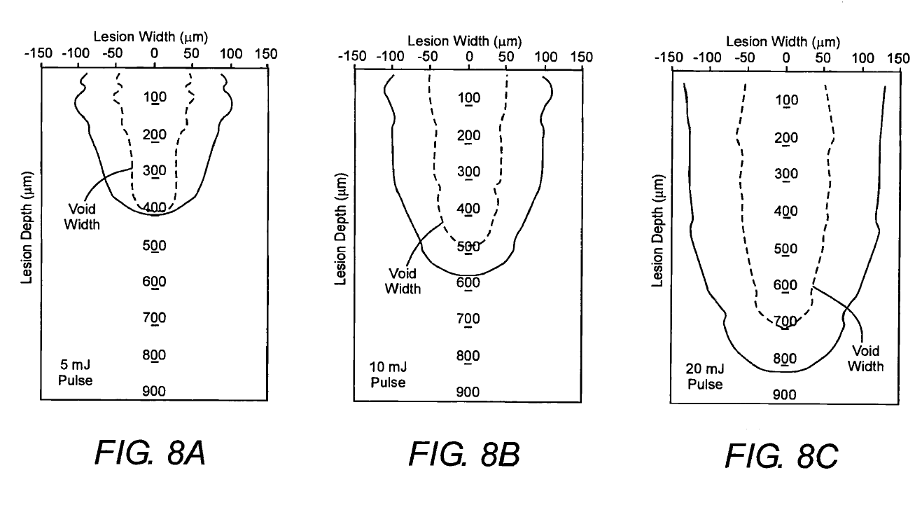

FIG. 8A, FIG. 8B, and FIG. 8C are graphs schematically illustrating graphical lesion width (solid curves) and void width (dashed curves) as a function of lesion depth for experimental irradiations at respectively 5 mJ, 10 mJ, and 20 mJ. These graphs are derived from measurements taken from micrographs of transverse sections through the experimental legions. The graphs of FIGS. 7 and 8A-C can be used as guidelines to select initial spacing of treatment zones in the inventive method. This spacing can then be optimized by experiment or otherwise.

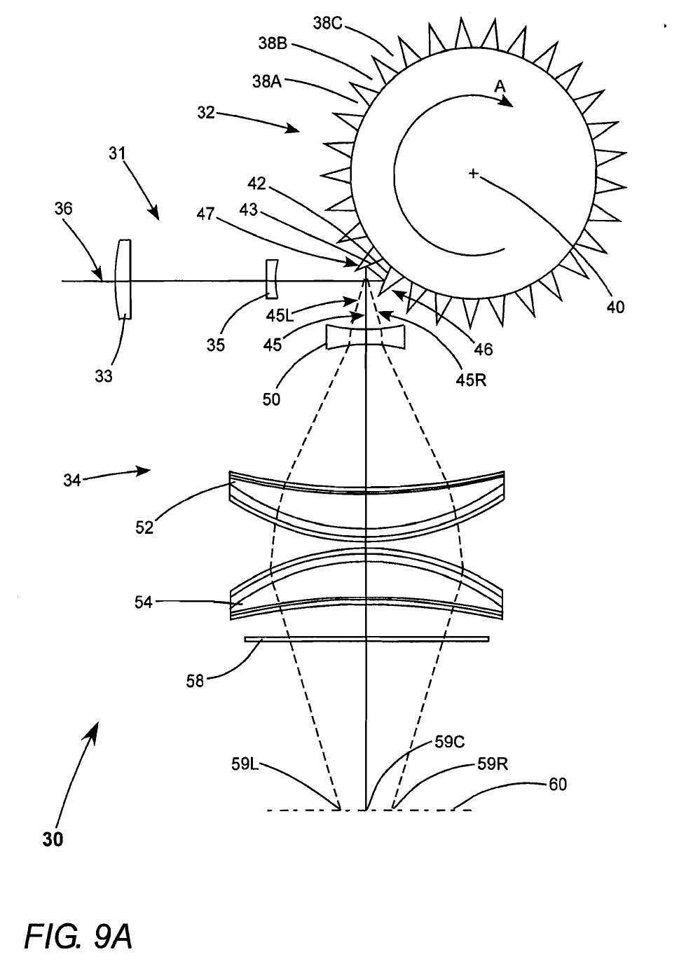

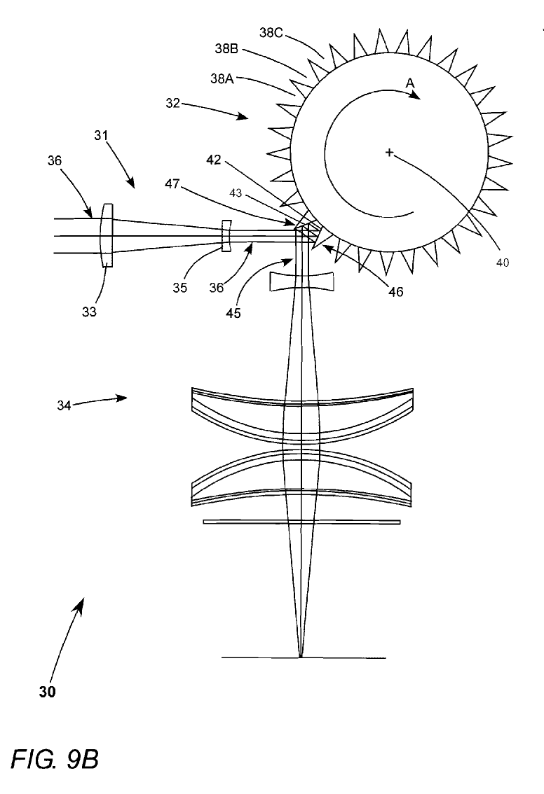

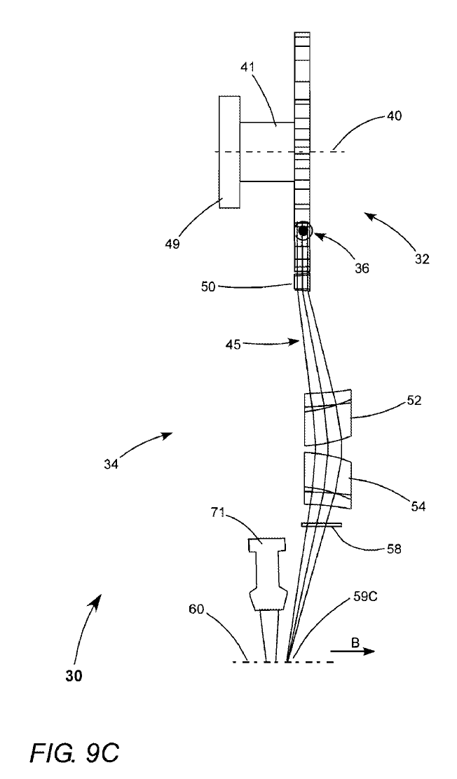

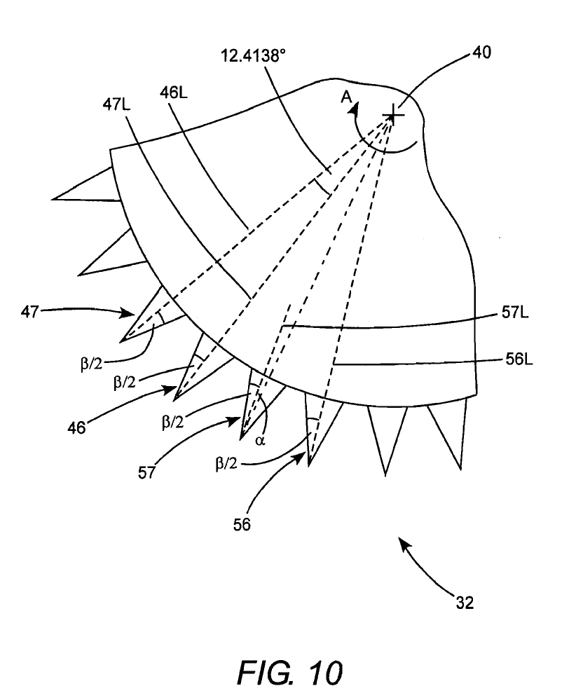

In any area being treated, all voids could be ablated simultaneously. However, apparatus capable of simultaneously ablating an effective number of voids with appropriate spacing over a useful area of skin may not be practical or cost effective. Practically, the voids can be ablated sequentially, but it is preferable that the area being treated, for example a full face, is completed in a time period less than about 60 minutes (min). It is preferable to create voids at a rate between about 10 Hz and 5000 Hz and more preferably at a rate between about 100 Hz and 5000 Hz, because this rate reduces the physician time for treatment. Increasing the treatment rate above 5000 Hz causes the laser and scanning systems to be more expensive and therefore less commercially desirable, even though they are technologically feasible using the apparatus presented here. One preferred example of apparatus for providing rapid sequential delivery of absorption pulses is described below with reference to FIG. 9A, FIG. 9B, FIG. 9C, and FIG. 10.

FIG. 9A is a front elevation view schematically illustrating ablation apparatus 30 including a scanner wheel 32 and a wide field projection lens 34. The scanner wheel is driven by a motor 49 via a hub 41 (see FIG. 9C). Scanner wheel 32 is arranged to receive an incident laser beam 36 lying substantially in the plane of rotation of the scanner wheel. In FIG. 9A beam 36 is represented by only a single principle ray. FIG. 9B and FIG. 9C are respectively front and side elevation views of apparatus in which beam 36 is represented by a plurality of rays.

Before being incident on the scanning wheel, beam 36 is compressed (see FIG. 9B) by a telescope 31 comprising a positive lens 33 and a negative lens 35. In this example, the scanner wheel divided into twenty nine sectors 38A, 38B, 38C, etc., which are arranged in a circle centered on the rotation axis 40 of the scanner wheel. The wheel, here, is assumed to rotate in a clockwise direction as indicated by arrow A. The incident laser beam 36 propagates along a direction that lies in the plane of rotation. Each sector 38 of scanner wheel 32 includes a pair of reflective elements, for example, reflective surfaces 42 and 43 for the sector that is indicated as being active. The surface normals of the reflective surfaces have a substantial component in the plane of rotation of the scanner wheel. In this example, the scanner wheel includes prisms 46, 47, etc. that are arranged in a circle. The faces of the prisms are reflectively coated and the reflectively coated surfaces of adjacent prisms, for example, reflective surfaces 42 and 43 from prisms 46 and 47, form the opposing reflective surfaces for a sector. Alternatively, the reflective surfaces can be metal surfaces that are polished to be smooth enough to cause sufficient reflectivity.

Each sector 38 deflects the incoming optical beam 36 by some angular amount. The sectors 38 are designed so that the angular deflection is approximately constant as each sector rotates through the incident optical beam 36, but the angular deflection varies from sector to sector. In more detail, the incident optical beam 36 reflects from the first reflective surface 42 on prism 46, and subsequently reflects from reflective surface 43 on prism 47 before exiting as output optical beam 45.

The two reflective surfaces 42 and 43 form a Penta mirror geometry. An even number of reflective surfaces that rotate together in the plane of the folded optical path has the property that the angular deflection of output beam 45 from input beam 36 is invariant with the rotation angle of the reflective surfaces. In this case, there are two reflective surfaces 42 and 43 and rotation of the scanner wheel 32 causes the prisms 46 and 47 and reflective surfaces 42 and 43 thereof to rotate together in the plane of the folded optical path. As a result, the output beam direction does not change as the two reflective surfaces 42 and 43 rotate through the incident optical beam 36. The beam can be focused at the treatment surface such that the beam does not walk across the surface during the scanning or the beam can be used at another plane such that the beam walks across the surface during the scanning due to the translation of the beam in a conjugate plane that translates into an angular variation during the scanning due to the rotation of the scanning wheel. The reflective surfaces 42 and 43 are self-compensating with respect to rotation of scanner wheel 32. Furthermore, as the reflective surfaces 42 and 43 are planar, they will also be substantially spatially invariant with respect to wobble of the scanner wheel.

As the scanner wheel rotates clockwise to the next sector 38 and the next two reflective surfaces, the angular deflection can be changed by using a different included angle between the opposing reflective surfaces. For this configuration, the beam will be deflected by an angle that is twice that of the included angle. By way of example, if the included angle for sector 38A is 45 degrees, sector 38A will deflect the incident laser beam by 90 degrees. If the included angle for sector 38B is 44.5 degrees, then the incident laser beam will be deflected by 89 degrees, and so on. In this example, different included angles are used for each of the sectors so that each sector will produce an output optical beam that is deflected by a different amount. However, the deflection angle will be substantially invariant within each sector due to the even number of reflective surfaces rotating together through the incident beam. For this example, the angular deflections have a nominal magnitude of 90 degrees and a variance of −15 to +15 degrees from the nominal magnitude. Beam 45 in extreme left and right scanning positions is indicated by dashed lines 45L and 45R respectively. Here again, in FIG. 9A beam 45 is represented by only a single principle ray, while FIG. 9B and FIG. 9C represent beam 45 by a plurality of rays.

Referring in particular to FIG. 10, in this example of scanner wheel 32, the apex angle of each prism is 32.5862 degrees, calculated as follows. Each sector 38 subtends an equal angular amount. Since there are twenty nine sectors, each sector subtends 360/29=12.4138 degrees. The two prisms 46 and 47 have the same shape and, therefore, the same apex angle β. Scanner wheel 32 is designed so that when the included angle is 45 degrees, the prisms 46 and 47 are positioned so that lines 47L and 46L that bisect the apex angle of prisms 46 and 47 also passes through the rotation axis 40. Accordingly, the design must satisfy an equation β/2+12.4138+β/2=45. Solving this equation yields an apex angle of β=32.5862 degrees.

The next prism 57 moving counterclockwise on scanner wheel 32 from prism 46 is tilted slightly by an angle +β so its bisecting line 57L does not pass through the center of rotation 40 of the scanner wheel. As a result, the included angle for the sector formed by prisms 46 and 57 is (β/2+α)+12.4138+β/2=45+α. The next prism 56 is once again aligned with the rotation center 40 (as indicated by bisecting line 56L), so the included angle for the sector formed by prisms 56 and 57 is (β/2−α)+12.4138+β/2=45−α. The next prism is tilted by +2 α, followed by an aligned prism, and then a prism tilted by +3α, followed by another aligned prism, etc. This geometry is maintained around the periphery of the scanner wheel. This specific arrangement produces twenty nine deflection angles that vary over the range of −15 degrees to +15 degrees relative to the nominal 90 degree magnitude. Note that this approach uses an odd number of sectors where every other (approximately) prism is aligned and the alternate prisms are tilted by angles α, 2α, 3α, etc. In an alternate embodiment, the surface on which beam 36 is incident has zero tilt and all tilt is taken up in the reflective surface on the second facet.

Wide field lens 34, here includes optical elements 50, 52, and 54, and an output window 58. In the lens depicted in FIGS. 9A-C the optical elements are assumed to made from zinc selenide which has excellent transparency for 10.6-micrometer radiation. Those skilled in the art will recognize that other IR transparent materials such as zinc sulfide (ZnS) or germanium (Ge) may be used for elements in such a lens with appropriate reconfiguration of the elements. Optical elements 52, 54, and 56 are tilted off axis spherical elements. Lens 34 focuses exit beam 45 from scanner wheel 32 in a plane 60 in which skin to be treated would be located. Lens 34 focuses exit beam 45 at each angular position that the beam leaves scanner wheel 32. This provides a line or row sequence of 29 focal spots (one for each scanning sector of the scanner wheel) in plane 60. In FIG. 9A three of those spots are designated including an extreme left spot 59L, a center spot 59C and an extreme right spot 59R. The remaining 26 spots (not shown) are approximately evenly distributed between spots 59L, 59C, and 59R. Another line of focal spots can be produced by moving apparatus 30 perpendicular to the original line as indicated in FIG. 9C by arrow B.

Referring in particular to FIG. 9C, the tilted off-axis spherical elements 50, 52 and 54 are arranged such that beam 45 is first directed, by (bi-concave negative) lens element 50, away from the plane of rotation of the scanner wheel. Elements 52 and 54 (positive meniscus elements) then direct the beam back towards the plane of rotation, while focusing the beam, such that the focused beam is incident non-normally (non-orthogonally) on plane 60, i. e., the surface of the skin being treated. One particular advantage of this non-normal incidence of beam 45 on the skin is that window 58 and optical element 54 are laterally displaced from the focal point and are removed from the principal path of debris that may be ejected from a site being irradiated. Another advantage is that a motion senor optics for controlling firing of the laser in accordance with distance traveled by the apparatus, for example, an optical mouse or the like, designated in FIG. 9C by the reference numeral 71, may be directed close to the point of irradiation. This is advantageous for control accuracy.

Those skilled in the art will recognize that is not necessary that all sectors of the scanner wheel have a different deflection angle. Prisms of the scanning wheel can be configured such that groups of two or more sectors provide the same deflection angle with the deflection angle being varied from group to group. Such a configuration can be used to provide fewer voids in a row with increased spacing therebetween. It is also not necessary that the deflection angle be increased or decreased progressively from sector to sector. It is preferred in that pulsed operation of the laser providing beam 36, that the PRF of the laser is synchronized with rotation of the scanner wheel such that sequential sectors of the wheel enter the path of beam 36 to intercept sequential pulses from the laser. Alternatively, a laser of sufficient power can be run in continuous wave (CW) mode, in which case, the scanner wheel effectively pulses the laser at sequential locations on the skin surface. This configuration reduces the complexity of the control electronics for the laser.

It should be noted here that apparatus 30 including scanner wheel 32 and focusing lens 34 is one of several combinations of scanning and focusing devices that could be used for carrying out the method of the present invention and the description of this particular apparatus should not be construed as limiting the invention. By way of example, different rotary scanning devices and focusing lenses are described in U.S. patent application Ser. No. 11/158,907, entitled “Optical pattern generator using a single rotating component” and filed Jun. 20, 2005, the complete disclosure of which is hereby incorporated by reference. Galvanometer-based reflective scanning systems can also be used to practice this invention and have the advantage of being robust and well-proven technology for laser delivery. Scanning rates with a galvanometer-based reflective scanning systems, however, will be more limited than with a scanner such as scanning wheel 32 described above, due to the inertia of the reflective component and the changes of direction required to form a scanning pattern over a substantial treatment area.

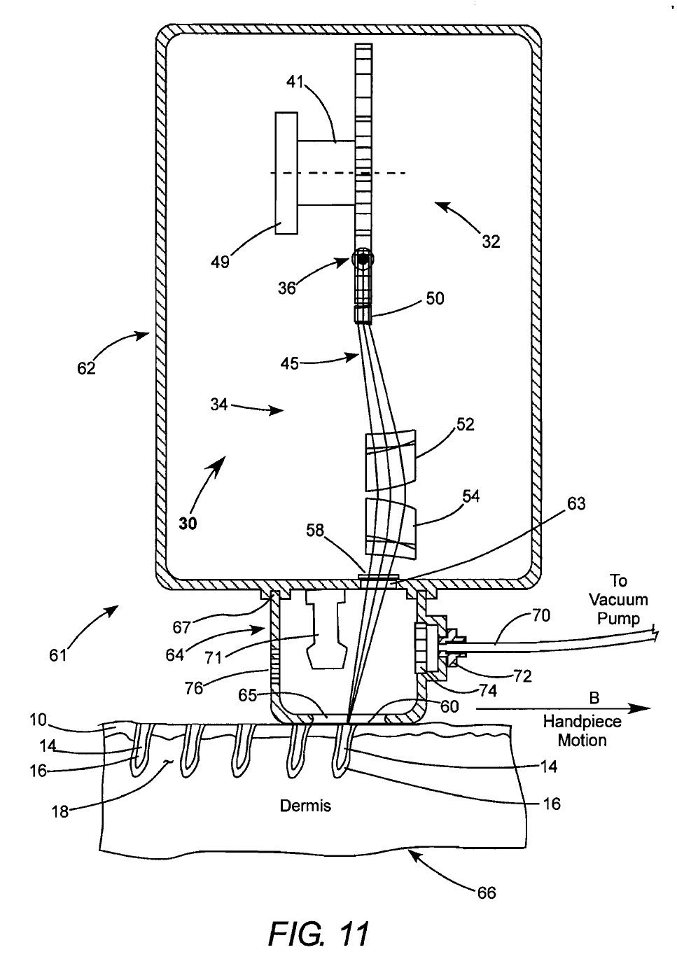

FIG. 11 schematically illustrates a handpiece 61 or applicator housing an example of above described apparatus 30. Handpiece 61 is depicted irradiating a fragment 66 of skin being treated. The handpiece is moved over the skin being treated, as indicated by arrow B, with tip 64 in contact with the skin. The irradiation provides parallel spaced-apart rows of above-described spaced-apart voids 14, only end ones of which are visible in FIG. 8. Spacing between the rows of spots may be narrower or broader than that depicted in FIG. 8, the spacing here being selected for convenience of illustration. Control of the row spacing can be affected by controlling delivery of the laser beam by optical motion sensor 71, or alternatively a mechanical motion sensor (mechanical mouse), as is known in the art. A description of such motion sensing and control is not necessary for understanding principles of the present invention and accordingly is not presented here. Descriptions of techniques for controlling delivery of a pattern of laser spots are provided in U.S. patent application Ser. No. 10/888,356 entitled “Method and Apparatus for fractional photo therapy of skin” and Ser. No. 11/020,648 entitled “Method and apparatus for monitoring and controlling laser-induced tissue treatment,” the complete disclosures of which are hereby incorporated herein by reference.

In a preferred method of operation, apparatus 30 is housed in handpiece or applicator 61 including a housing 62 to which is attached an open-topped, removable tip 64, which is attached to the housing via slots 67. Pins and/or screws can also be used for this purpose. Laser beam 36 is directed into housing 62 via an articulated arm (not shown). Articulated arms for delivery of infra red laser radiation are well known in the art. One preferred articulated arm is described in U.S. patent application No. 60/752,850, filed Dec. 21, 2005 and entitled “Articulated arm for delivering a laser beam,” the complete disclosure of which is hereby incorporated herein by reference. The focused beam 45 from lens 34 exits housing 62 via exit window 58, (here attached to the housing) and via aperture 63 in the housing, then passes through tip 64 exiting via aperture 65 therein. A vacuum pump (not shown) is connected to removable tip 64 via a hose or tube 70. Tube 70 is connected to tip 64 via a removable and replaceable adaptor 72. Operating the vacuum pump with tip 64 in contact with the skin creates negative pressure (partial vacuum) inside the tip. This draws air into the tip, via apertures 76 therein, and serves to create an air-flow through the tip, withdrawing smoke resulting from the laser ablation from the path of the laser beam, and drawing debris products of the ablation away from window 58 in the housing. A filter element 74 in a wall of tip 64 prevents debris from being drawn into vacuum hose 70 and eventually into the pump. One skilled in the art will recognize, without further illustration that hose 70 could be connected to an air pump or compressed gas supply such that an air flow through the tip could created by forcing air through the tip exiting via apertures 76 therein.

Even with the preventive measures described above, some contamination of window 58 may be inevitable. Further, filter element 74 can become blocked by debris to an extent that pumping of the tip is compromised. Such problems can be corrected in a number ways. By way of example can be removed and replaced with a new tip, or filter 74 can be replaced. When tip 64 is removed, window 58 in the housing can be either cleaned or replaced. One method for facilitating cleaning of window 58 would be to cover the window with a stack of layers of a transparent foil. When the window becomes contaminated to the point at which cleaning is required the outer, contaminated, layer can be removed from the stack to expose a clean layer. Those skilled in the art may devise other contamination reducing methods or devices without departing from the spirit and scope of the present invention.

The arrangement of apparatus 30 in handpiece 61 is but one possible arrangement for providing nonorthogonal incidence of the focused beam on the skin surface. Those skilled in the art may devise other arrangements without departing from the spirit and scope of the present invention. By way of example, the laser beam could be tilted by an optical component such as mirror or prism located in housing 62, or located in tip 64 after delivery from lens 34. It should be noted however that any such optical component located in tip 64 could itself become contaminated by debris.

While the laser irradiation method of the present invention is described above in terms of a method for tightening skin to reduce the appearance of wrinkles, the healing process by which the skin tightening is effected makes the irradiation method useful for treating other skin conditions. One such condition is melasma. Melasma is a dark skin-coloration found on sun exposed areas of the face. Melasma can affect anyone. However, young women with brownish skin tones are at greatest risk. Melasma is often associated with the female hormones estrogen and progesterone. It is especially common in pregnant women, women who are taking oral contraceptives, and women taking hormone replacement therapy during menopause. Sun exposure is also a strong risk factor for melasma. Melasma doesn't cause any other symptoms besides skin discoloration but may be of great cosmetic concern. A uniform brown color is usually seen over the cheeks, forehead, nose, or upper lip. This is due to a preponderance of melanin containing cells in the affected areas. This method is particularly appropriate because dermal and epidermal melanin can be ejected from the skin while not stimulating an excessive inflammatory response. This method is particularly suited for treatment of melasma that includes a dermal component. Such melasma is difficult to treat by other typical modalities, such as bleaching creams.

For the treatment of such a condition more than one cycle of irradition and subsequent healing would be required to completely eliminate the condition, as in any one cycle of fractional ablation and healing only the coagulated regions surrounding ablated voids is replaced by new collagen and elastin. New dermal collagen and elastin would not contain abnormal amounts of melanin. Additionally, other cells around each void can benefit from the wound healing process that is stimulated.

Another use for a fractional ablative laser is the retexturing of scars. Drilling holes with an ablative laser can also be used to retexture skin by creating new rete ridge-like structures for retexturizing scars after the tissue has healed. The invention has the advantage of removing some of the scar tissue and allowing the surrounding viable tissue to heal the coagulated area with new, viable, normal skin. The ablative treatment described in this invention allows removal of scar tissue more effectively than nonablative treatments and treats deeper for a similar number of side effects than other ablative treatments. The inflammation from the acute wounds created by the fractional treatment would also possibly disrupt the abnormal synthesis:destruction cycles for the collagen within the scar. Alternatively, the CO₂ laser could be used to burn dermatoglyphs into the scar to create texture in a particular pattern that matches surrounding tissue. Striae, or stretch marks, could also be retextured with this method.

Texturing could also be used to help skin grafts or implants “take” better. Fractional ablative treatment could also be used in the area surrounding an incision after surgery to provide better healing for incisions and to reduce the chance of scarring. This improvement would occur due to the controlled stimulation that would be provided by the ablative treatment. The fractional ablative treatment could be done at the time of sewing the skin together or in the period of 1 to 6 weeks following surgery after the wound has had time to get beyond its initial trauma due to surgery.

The following concerns results from a clinical study of laser irradiation in accordance with a method of the present invention.

A study protocol was approved by an institutional review board and all subjects were consented prior to participation in the study. Twenty four healthy subjects of Fitzpatrick skin types II-IV were treated on the forearm with a 30 W, CO₂ laser system to assess the wound healing response of human skin in-vivo, post treatment. The CO₂ laser system has a beam quality with an M² value of less than 1.2. The laser beam was delivered through multiple deflective and refractive elements and focused to a series of discrete locations with a diffraction-limited 1/e² spot size (diameter) of approximately 120 μm at the skin surface.

Topical anesthesia was administered locally prior to laser treatment. The forearm of each subject was first cleansed with alcohol, after which a 23% lidocaine, 7% tetracaine ointment was topically applied on the intended treatment sites and occluded for approximately 30-45 minutes. The topical anesthesia was wiped off before the treatment was administered. The laser handpiece was moved at a constant velocity across the subject's forearm and the handpiece was configured to allow deposition of a constant density of microscopic treatment zones (MTZs) as the handpiece moved across the skin. Each laser treatment site comprised a skin area of approximately 1.5 cm by 1.0 cm. Pulse energies ranged from about 5 to 40 mJ. Treatments were performed in a single pass with a spot density of about 400 MTZ/cm² for pulse energies 5-30 mJ and 100 MTZ/cm² for pulse energy of 40 mJ. A total of twenty-four subjects received multiple treatments at varying pulse energies prior to biopsy excisions that were made immediately, 2 days, 7 days, 1 month, and 3 months post-treatment. The biopsy schedule is outlined in Table 1.

Immediately following excision, each biopsy sample was fixed in 10% v/v neutral buffered formalin (VWR International, West Chester, Pa.) overnight and then embedded in paraffin. The samples were sectioned into slices that were approximately 5-10 μm thick. The slices were stained with hematoxylin and eosin (H&E), hsp72 antibody, or hsp47 antibody. A minimum of ten lesions from the histological sections of each biopsy sample were imaged and recorded using a Leica® DM LM/P microscope and a DFC320 digital camera (Leica Microsystem, Cambridge, United Kingdom). Lesion dimensions were measured based on the H&E stained slices. The lesion dimensions reported in Table 2 represent the maximum depth and width of the outermost border of the coagulation zones for these experiments.

TABLE 1
Number of biopsies excised and number of lesions
evaluated at various time points as part of in
vivo laser irradiation clinical study.

| Energy | | | | | |
|---|---|---|---|---|---|
| per | Biopsy time following laser irradiation | | | | |
| treatment | # of biopsies (n = # of lesions evaluated) | | | | |
| zone (mJ) | <1 hour | 2 days | 7 days | 1 month | 3 months |
| 5 | 6 (n = 60) | 6 (n = 60) | — | 1 (n = 10) | — |
| 10 | 7 (n = 70) | — | 3 (n = 30) | — | — |
| 20 | 2 (n = 20) | 5 (n = 50) | 3 (n = 30) | 2 (n = 20) | 2 (no visible |
| | | | | | lesions) |
| 30 | 3 (n = 30) | — | — | 2 (n = 20) | — |
| 40 | — | — | — | — | 2 (no visible |
| | | | | | lesions) |

TABLE 2
Lesion dimensions measured in clinical study for lesions
excised within 1 hour following in vivo laser irradiation

| Energy | Maximum | Maximum | Maximum | Maximum | Thickness |
|---|---|---|---|---|---|
| per | Lesion | Lesion | Ablative | Ablative | of |
| treatment | Depth | Width | Depth | Width | coagulated |
| zone (mJ) | (μm) | (μm) | (μm) | (μm) | zone (μm) |
| 5 | 298 ± 48 | 138 ± 20 | 210 ± 67 | 71 ± 17 | 33 ± 11 |
| 10 | 439 ± 70 | 184 ± 15 | 286 ± 76 | 95 ± 17 | 44 ± 13 |
| 20 | 778 ± 57 | 218 ± 10 | 560 ± 86 | 110 ± 18 | 54 ± 8 |
| 30 | 993 ± 77 | 270 ± 23 | 659 ± 69 | 121 ± 16 | 75 ± 13 |

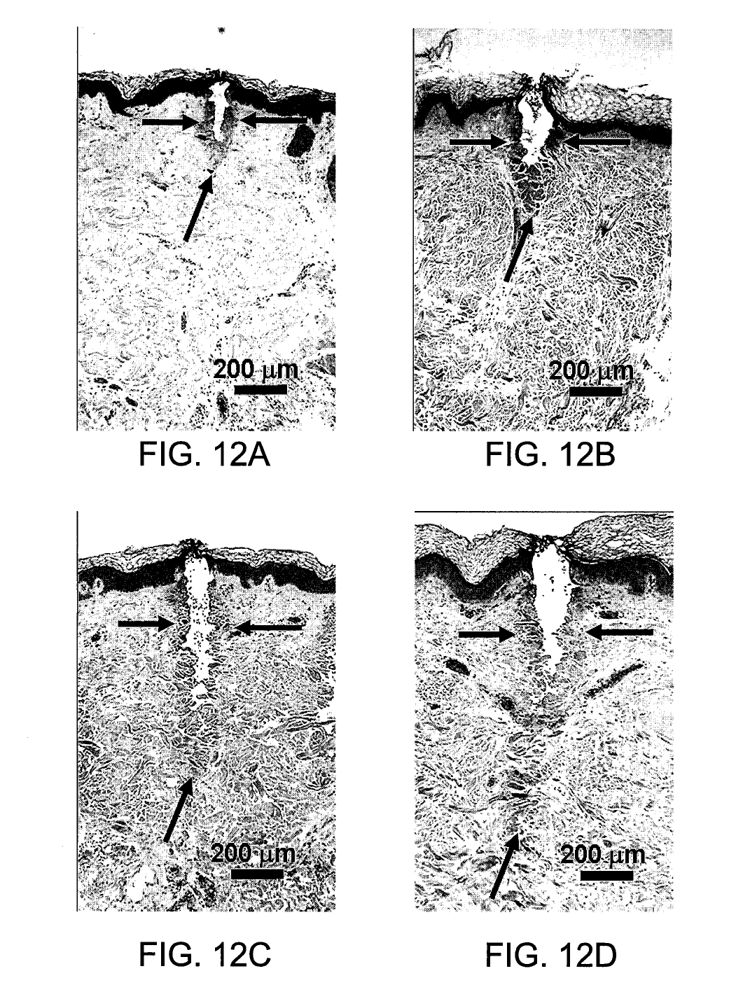

FIGS. 12A-D show treatment zones for biopsy sections that were excised within approximately 1 hour following laser irradiation with laser radiation having parameters in accordance with the method of the present invention. Each section was stained with hemotoxylin and eosin (H&E). FIGS. 12A-D show results treating with different treatment pulse energies: FIG. 12A depicts treatment zones created using a pulse energy of 5 mJ, whereas FIGS. 12B, 12C, and 12D show treatment zones created using pulse energies of 10 mJ, 20 mJ, and 30 mJ, respectively. As shown by comparing FIGS. 12A-D, the depth and width of ablative zones can be adjusted by altering the pulse energy. Each ablative zone was surrounded by a layer of coagulation zone that promoted hemostasis and tissue shrinkage.

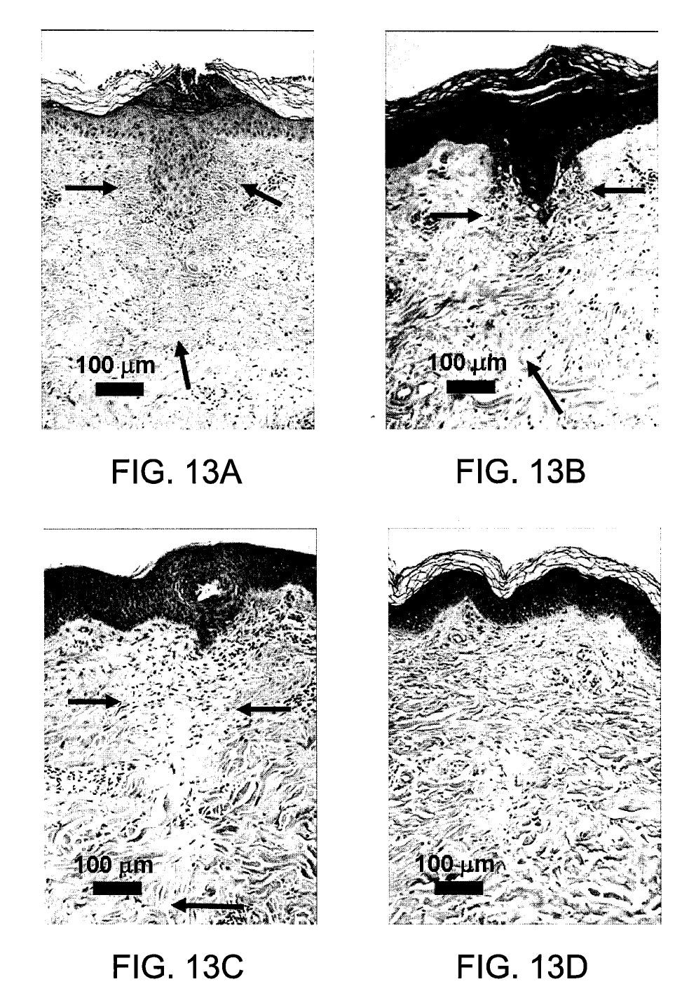

FIGS. 13A-D show treatment zones for H&E stained biopsy sections that were excised approximately 2 days, 7 days, 1 month, and 3 months following laser irradiation with laser radiation having parameters in accordance with the method of the present invention. The histology images show aspects of the process of wound healing with invagination of the epidermis into the ablative zone. Complete re-epithelialization occurred within 2 days of irradiation. A sustained coagulation zone was still demarcated in the sections excised 1 month following laser irradiation, which indicates that a long-term remodeling process is occurring. A regressed epidermal invagination with replacement of new collagen within the original ablative zone was observed in biopsies of sections taken 1 month and 3 month post irradiation.

As shown in the H&E-stained image in FIGS. 12A, the laser irradiation led to immediate ablation of the epidermis and dermis. The tapering shape of ablative zones excised within 1 hour of irradiation ranged from 71 to 121 μm in width and 210 to 659 μm in depth for the pulse energies of 5 to 30 mJ. The ablative zone was lined by a thin layer of eschar, and on occasion contained a serum exudate and red blood cells, none of which was found to be extravasated in the dermis as shown in FIG. 12A. Our histology results suggest that adequate hemostasis was achieved with the selected parameters across the range of tested treatment energies, partially due to the surrounding thermal coagulation zone (33 to 75 μm in thickness). For the pulse energies tested, the representative lesions measured about 138 to 270 μm in width and about 298 to 993 μm in depth with an interlesional distance of approximately 500 μm, as described in more detail in Table 2.

To promote healing, the cross sectional area of the voids can be limited to the range of about 0.01 to 1.0 mm², or about 0.03 to 0.5 mm², or about 0.1 to 0.2 mm². In some embodiments, the voids created according to the invention can be into the reticular dermis to create deep dermal remodeling and tightening of deep dermal layers. In these cases, the cross sectional area of the voids can still be limited in order to promote healing. In these cases, the ratio of the cross sectional area of the void to the depth of the void can be in the range of 0.01 to 2 mm, or about 0.05 to 0.5 mm, or 0.1 to 0.5 mm. Alternatively, the diameter of the void to the depth of the void can be in the range of about 0.05 to 1.0 or about 0.1 to 0.5.

For the samples viewed 2 days following irradiation, the ablative zone was completely replaced by invaginating epidermal cells as illustrated in FIG. 13A. The MTZ surrounded the newly invaginated epidermal tissue, although the basement membrane remained partially disrupted as evidenced by basal layer vacuolar change.

By 7 days post-treatment, exfoliation was evident with residual material at the very superficial aspect of the stratum corneum as shown in FIG. 13B. H&E staining of the coagulation zone appeared diminished, but close inspection of the dermis revealed an increase in the number of spindle cells; this suggests the continued presence of fibroblast activity, consistent with ongoing dermal remodeling.

By 1 month post-treatment, the stratum corneum appears normal and residual material was no longer detectable in the stratum corneum. The epidermal invagination had significantly regressed, as shown in FIG. 13C. In addition, the space vacated by the regressed epidermis within the MTZ was replaced by newly synthesized collagen. H&E staining of the coagulation zone surrounding the original ablative zone was diminished but relatively well demarcated, indicating a slow but continuous dermal remodeling process. Both collagen within the original ablative zone and within the coagulation zone appeared haphazard. Spindle cells remained abundant around and especially within the dermal zone of thermal coagulation at this stage.

At 3 months post-treatment, H&E staining showed no definitive evidence of micro-lesions, with only rare areas in the dermis resembling ‘old’ lesions, as shown in FIG. 13D.

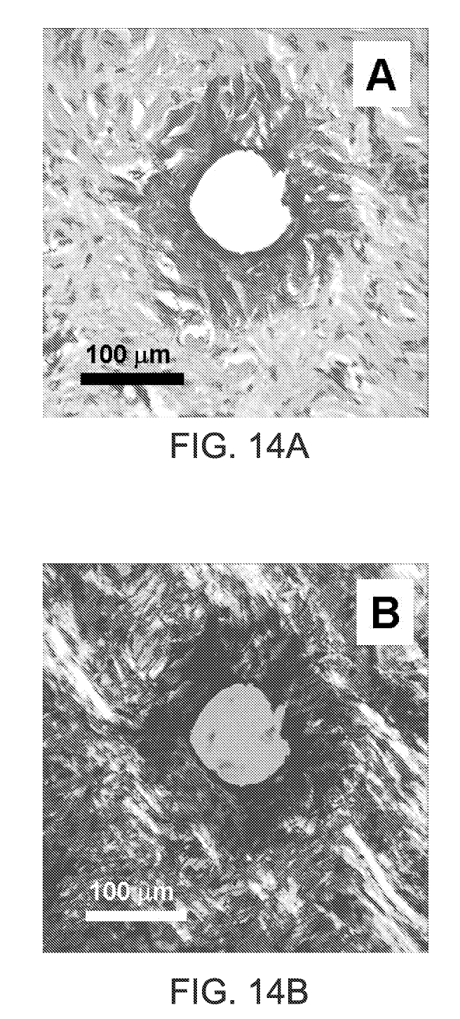

FIG. 14A depicts a horizontal cross-section of a lesion created by ablative laser irradiation. The horizontal cross-section is from a depth of approximately 350 μm beneath the surface of the skin. A clear zone of annular collagen denaturation was observed surrounding the microlesion. This was confirmed by a cross-polarized image of the same lesion as shown in FIG. 14B, with loss of birefringence within the collagen denaturation (coagulation) zone.

In summary, the present invention is described above in terms of a preferred and other embodiments. The invention is not limited, however, to the embodiments described and depicted. Rather, the invention is limited only by the claims appended hereto.

What is claimed is:

1. A method of treating skin, the skin characterized as having a stratum corneum surmounting an epidermis, the epidermis surmounting a dermis, the method comprising:
irradiating the skin with laser radiation in a manner such that a plurality of elongated spaced-apart voids are formed in the skin, said voids extending through the stratum corneum, through the epidermis, and into the dermis, with a volume of coagulated dermal tissue surrounding the voids, and with viable tissue remaining between the coagulated-tissue-surrounded voids, and such that shrinkage of collagen in the coagulated tissue surrounding the voids causes an essentially immediate reduction of the volume of the voids, and a subsequent healing process eliminates the voids and replaces coagulated tissue with new tissue.

2. The method of claim 1, wherein the skin is wrinkled skin and the treatment reduces the wrinkling of the skin.

3. The method of claim 1, wherein the skin is abnormally pigmented skin and the treatment reduces the abnormal pigmentation.

4. The method of claim 3, wherein the abnormal pigmentation is natural.

5. The method of claim 4, wherein the abnormal pigmentation results due to melasma.

6. The method of claim 1, wherein the skin is scar tissue.

7. The method of claim 1, wherein the laser radiation has a wavelength that is strongly absorbed by water.

8. The method of claim 7, wherein the laser radiation has an absorption coefficient in water is between about 100 cm⁻¹ and 12,300 cm⁻¹.

9. The method of claim 8, wherein the absorption coefficient is between about 100 cm⁻¹ and 1000 cm⁻¹.

10. The method of claim 9, wherein the absorption coefficient is between about 500 cm⁻¹ and 1000 cm⁻¹.

11. The method of claim 1, wherein the laser radiation is delivered as a sequence of pulses with each pulse forming a corresponding one of said spaced-apart voids.

12. The method of claim 11, wherein said laser radiation is delivered as a sequence of pulses at a pulse repetition rate of between about 100 and 5000 Hz.

13. The method of claim 1, wherein each of said laser radiation is delivered in a beam thereof inclined orthogonally to the skin.

14. The method of claim 2, wherein each of said pulses is delivered in a beam thereof inclined non-orthogonally to the skin.

15. The method of claim 1, wherein the density of spaced-apart voids is about 200 and 5000 treatment zones per cm².

16. The method of claim 1, wherein the density of spaced-apart voids is about 1000 and 3000 treatment zones per cm².

17. A method of treating skin laxity, the skin characterized as having a stratum corneum surmounting an epidermis, the epidermis surmounting a dermis, the method comprising:
delivering a plurality of pulses of laser radiation from a CO₂ laser to the skin in a manner such that a plurality of elongated spaced-apart voids are formed in the skin, said voids extending through the stratum corneum, through the epidermis, and into the dermis, with a volume of coagulated dermal tissue surrounding the voids, and with viable tissue remaining between the coagulated-tissue-surrounded voids, and such that shrinkage of collagen in the coagulated tissue surrounding the voids causes a prompt immediate shrinkage of the voids causing a corresponding reduction of the skin laxity, and a subsequent healing process eliminates the voids and replaces coagulated tissue with new tissue.

18. The method of claim 17, wherein said CO₂ laser radiation has a wavelength of about 10.6 micrometers.

19. The method of claim 18 wherein said voids have a cross sectional area at the skin surface of between about 0.02 mm² and 0.5 mm².

20. The method of claim 18 wherein for one or more of said voids, the ratio of the cross sectional area in the plane of the skin surface of the one or more said voids to the depth of the one or more said voids is in the range of between about 0.01 mm and 2 mm.

21. The method of claim 18 wherein said voids have a diameter between about 100 micrometers and 500 micrometers and a depth between about 200 micrometers and 4.0 millimeters.

22. The method of claim 19 wherein for one or more of said voids, the ratio of the diameter of the one or more said voids to the depth of the one or more said voids is in the range of about 0.05 to 1.0.

23. The method of claim 19, wherein the thickness of coagulated tissue surrounding a said void is about 20 micrometers and 80 micrometers immediately after ablation of the voids.

24. The method of claim 23, wherein said voids and coagulated tissue initially cover between about 5 percent and 50 percent of an area of skin irradiated by said laser radiation pulses.

25. The method of claim 24, wherein said radiation pulses have an energy between about 5 millijoules and 40 millijoules.

26. The method of claim 17, wherein said voids are randomly distributed over an area of skin irradiated by said laser radiation pulses.

27. The method of claim 17, wherein said delivering of a plurality of pulses of laser radiation from a CO₂ laser to the skin is performed within one hour following the creation of an incision in the skin.

28. The method of claim 17, wherein said delivering of a plurality of pulses of laser radiation from a CO₂ laser to the skin is performed during the period 1 to 6 weeks following the creation of an incision in the skin.

29. Apparatus for laser treatment of skin, the laser treatment including irradiating the skin with laser radiation in a manner such that a plurality of elongated spaced-apart voids are formed in the skin, the apparatus comprising:
a housing;
a scanning apparatus located in said housing, said scanning apparatus arranged to receive a the laser radiation and arranged to reflect the laser radiation in a plurality of different directions;
a lens located in said housing, said lens arranged for focusing the laser radiation at a plurality of different points laterally spaced in a focal plane with spacing corresponding to the plurality of different directions of reflection; and
a tip removably attached to said housing for making contact with the skin, said tip having optical access to said housing and optical access to the skin for allowing passage through said tip of the laser radiation focused by said lens, said optical access of said tip to the being about in said focal plane.

30. The apparatus of claim 29 wherein said tip includes a port connectable to a vacuum pump for exhausting smoke resulting from the ablation from the tip.

31. The apparatus of claim 30 wherein said tip has a plurality of apertures therein for allowing a flow of air through said tip from said apertures to said port.

32. The apparatus of claim 30, wherein said tip has a replaceable filter therein covering said port preventing ingestion of debris via said port into said vacuum pump.

33. The apparatus of claim 29, wherein said tip includes a port connectable to an air pump and has a plurality of apertures therein for providing a flow of air through said tip from said port to, said apertures for allowing ejection of smoke, resulting from the ablation, from the tip.

34. The apparatus of claim 29, wherein the optical access of said to tip to said housing includes a window transparent to the laser radiation and arranged to prevent air-flow between the tip and the housing.

35. The apparatus of claim 34, wherein said optical access of said tip to said housing includes an aperture in said housing, and said window is attached to said housing covering said aperture.

36. The apparatus of claim 34, wherein said window, on a side thereof facing into said tip has a plurality of layers of material thereon transparent to the laser radiation, said layers being sequentially removable one from the other.

37. The apparatus of claim 36, wherein said layers are plastic layers.
